(19) United States
(12) Patent Application Publication
(10) Pub. No.: US 2023/0223119 A1
(43) Pub. Date: Jul. 13, 2023

(54) Method for Managing Personal Health Information, Software and System for Same

(71) Applicant: OtisHealth, Inc., Cupertino, CA (US)

(72) Inventor: Marc Mar-Yohana, Cupertino, CA (US)

(21) Appl. No.: 17/891,115

(22) Filed: Aug. 18, 2022

Related U.S. Application Data
(60) Provisional application No. 63/235,078, filed on Aug. 19, 2021.

(51) Int. Cl. G16H 10/60

(52) U.S. Cl. CPC G16H 10/60

(57) ABSTRACT

A computerized method for analyzing healthcare data of a user that comprises uploading electronic healthcare data of the user into a system that includes a first plurality of healthcare events of the user. A second plurality of healthcare events of the user is manually entered into the system. Each event in the first and second plurality of healthcare events includes at least a date, medical data pertaining to the user and a source of the medical data. Each event in the first and second plurality of healthcare events is assigned to at least one medical category and the events in each medical category are ordered by date. The aggregation of electronic and manual healthcare events and chronological ordering of the events facilitates diagnosis and treatment of the user.

### CROSS REFERENCE TO RELATED APPLICATION

This application claims priority to U.S. provisional patent application Ser. No. 63/235,078 filed Aug. 19 2021, the entire content of which is incorporated herein by this reference

### FIELD OF THE INVENTION

This invention is in the field of health and relates more specifically to personal health data, personal healthcare records, personal health insights, and communication between healthcare organizations, patients, and community service organizations.

### BACKGROUND OF THE INVENTION

Nearly all personal health data is collected and retained by healthcare providers. In most cases, the data is limited to periodic check-ups or visits to treat illness. This intermittent data collection makes it difficult to establish a baseline of key health parameters such as vitals. It also means that moderate trends or changes in health may go unnoticed, thus potentially missing an opportunity to prevent a serious illness. There is a need for a complete, chronological record of all health data of a patient so as to provide substantial improvement to illness detection and prevention.

### BRIEF DESCRIPTION OF THE DRAWINGS

The drawings herein are schematic and not drawn to scale and are for illustration purposes only and are not intended to limit the scope of the present disclosure.

FIG. 1—Sample Application Environment showing system resources as rounded boxes with dashed lines. Sample elements of the platform of the invention are shown with solid line rounded boxes. Databases are shown as cylindrical shapes.

FIG. 2—Sample Health Profile features a dashboard of modules including check-mark symbols and percentage complete indicators for each module indicating completion status.

FIG. 3—Sample Vitals module showing graphed view of Body Weight parameter.

FIG. 4—Sample Add Medication window within the Medications & Supplements module, showing the search box functionality.

FIG. 5—Sample Add Medication window showing the auto-fill of BRAND NAME, GENERIC NAME, and MANUFACTURER NAME fields.

FIG. 6—Sample of medication data that has been uploaded (Benzodiazepines), showing the “SET REMINDER” feature.

FIG. 7—Sample Add Vaccinations/Immunizations window within the Vaccinations/Immunizations module, showing the search box functionality.

FIG. 8—Sample Allergies & Sensitivities module showing both entered and retrieved records. Former or inactive records can be shown in a suitable color.

FIG. 9—Sample Medical Conditions sections showing top selections left and bottom selections right.

FIG. 10—Sample Medical Conditions module showing a sample section “Heart, Arteries, Veins, and Circulatory System” with relevant condition option selections. Section completion status can be shown on the left scrolling list feature, for example as a “0%” icon or a check-mark symbol.

FIG. 11—Sample window that prompts user to provide more detail for a selected medical condition.

FIG. 12—Sample display showing both manually entered and uploaded medical condition records, both under the medical condition section. In this example, the “Age-Related Macular Degeneration” condition was read from an uploaded CCDA file and mapped to the correct Medical Condition section using the capability of the platform of the invention to automatically map clinical data using standard clinical codes.

FIG. 13—Sample Lab Work & Test Results module showing continuous list of test results and document upload option.

FIG. 14—Sample Surgeries and Procedures module showing manually entered, uploaded, and retrieved records on one screen.

FIG. 15—Sample Add Surgeries and Procedures window showing option to upload documents related to this record.

FIG. 16—Sample Clinical Visits and Hospitalizations module showing manually entered, uploaded, and retrieved records on one screen.

FIG. 17—Sample Add Clinical Visits and Hospitalizations window showing option to upload documents related to this record.

FIG. 18—Sample Family health history can be entered using a checkbox table. With each checkbox, a pop-up window can give users the ability to add detail. Users can modify the table to add more family members and Diseases and Conditions.

FIG. 19—Sample Personal Assessment module showing two depression screening tools with trackable scoring. The Personal Health Questionnaire (top) can for example be for users over the age of 18 and the Children's Happiness Scale (bottom) can for example be for users under the age of 18.

FIG. 20—Sample Health Insights can be automatically generated for each user based on health data they entered, upload, or retrieved. This may be augmented with data collected directly from wearable technology and other integratable health data capture technology.

FIG. 21—Sample Sharing feature showing the status of data shared with other users.

FIG. 22—Sample Sharing window showing options for granting edit or view only access to users. Window can also show how users can pick specific modules and features to share.

FIG. 23—Provide and Insurance feature showing provider screen.

FIG. 24—Sample Window showing the ability to add Healthcare Providers. This widow can also display the search tool which validates and retrieves the provider's profile data from the National Plan and Provider Enumeration System (NPPES) registry.

FIG. 25—Sample user view of My Communities and Join Communities options.

FIG. 26—Sample request new community capability.

FIG. 27—Sample of a community page.

FIG. 28—Sample create a reminder window showing configurable timing options.

FIG. 29—Sample on-screen activity reminder with COMPLETE and DISMISS options

FIG. 30—Sample Activities & Reminders feature showing Historical Activities log.

FIG. 31—Sample window showing list of organizations available to select for retrieval of electronic health records.

FIG. 32—Sample window for user to initiate a request for a CCDA file from a provider.

FIGS. 33A and 33B—Sample table of mapping of ICD chapter code range to modules and sections of the platform of the invention with both platform and ICD descriptions.

FIG. 34—Sample user profile management screen showing additional profiles being managed at the top right of the window.

FIG. 35—Sample window showing a warning message for the primary profile owner when converting a managed profile into an account.

FIG. 36—Four sample screens of the mobile device application with features common to the web-based application.

FIG. 37—Sample integration of 3rd party applications and devices.

FIG. 38—Sample screens showing the types of information that can be coordinated between Healthcare Providers, Community Service Organizations, and Users using the Community Support Services Coordination Feature.

FIG. 39—Process of a User using the platform of the invention to respond to a request from a Healthcare Provider for their health records.

FIG. 40—Alternative process of user using the mobile app and platform to grant a Healthcare Provider access to their health records using a QR code and a web page.

### DETAILED DESCRIPTION OF THE INVENTION

The platform of the invention, which can also be referred to as a system, is a combination of software and hardware designed to provide a service that users can access through internet web browsers and mobile devices. The platform can provide the ability to create, upload, retrieve, store, view, edit, and share personal health information. The platform can provide personal healthcare reminders, health analytics, and personalized insights based on users' interests and information provided. The platform can allow for the retrieval of medical records from healthcare providers via electronic record upload and file transfer through computer networks. The platform can allow for the upload, viewing, and download of images, scanned documents, and electronic documents. The platform can allow integration between the platform's mobile device application and other devices such as smart watches, pulse oximeters, blood pressure cuffs, blood glucose monitors and any other suitable wearable device. The integration with other devices can allow the platform to automatically collect and store user health information that is generated by those other devices.

Personal health records, health information and health data collected from all sources can be combined and time-sequenced to create a single, continuous, chronological record for each user. Furthermore, the record can by organized and referenceable as a subsection of a continuous health record. Each subsection in turn can have the combined data from all sources time-sequenced to create a referenceable, chronologically sorted health subsection. Examples of health record subsections can include vitals, lab results, medical conditions, medications, and any subcomponent of a health record.

Personalized health insights can be delivered to users with the application via graphs showing health trends and written messages.

### Application Environment

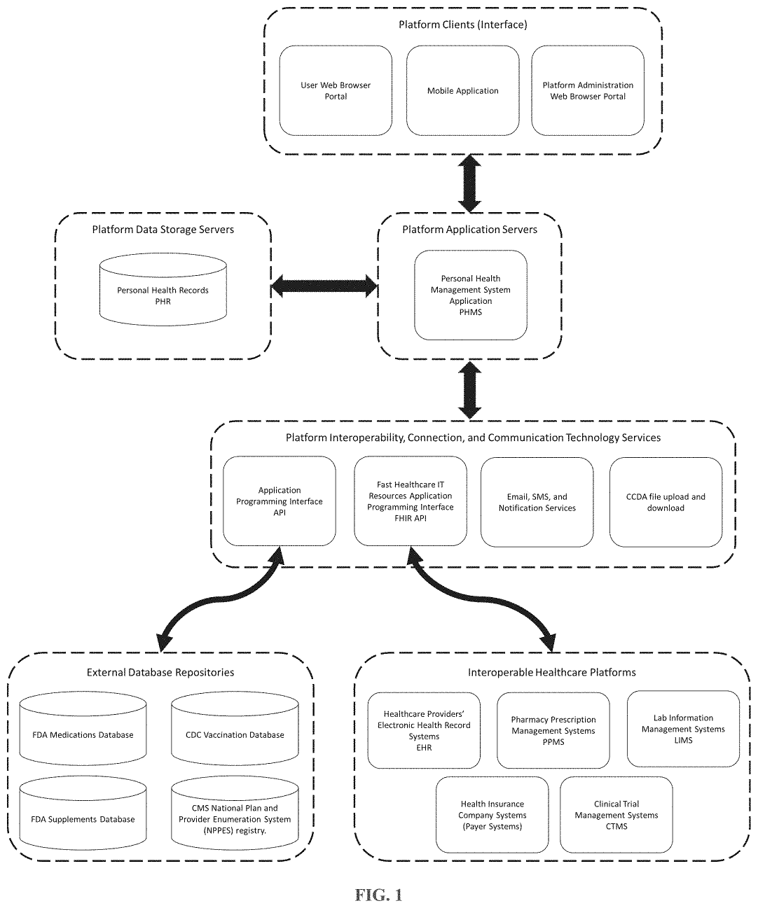

The platform of the invention can be hosted on a cloud-based server environment, for example as shown in FIG. 1. This environment can include servers that manage access for users and system administrators, servers that run the Personal Health Management System (PHMS) software application and provide computing microprocessors, data storage servers, and hardware to allow the platform to communicate with other systems via application programmable interfaces (APIs). The APIs can include connections to:
Food and Drug Administration (FDA) databases for data on approved medications and registered supplements.
FDA and Centers for Disease Control and Prevention (CDC) databases for data on approved vaccines.
Centers for Medicare and Medicaid Services (CMS) for data on register healthcare providers.
Healthcare providers' Electronic Health Record systems (EHR) for retrieval of user health records.
The record management systems of health insurance companies (also known as Payers) for retrieval of user coverage and claims history.
Pharmacy Prescription Management Systems (PPMS) for retrieval of users' history of prescribed and filled medications.
Laboratory Information Management Systems (LIMS) for retrieval of user lab results.
Clinical Trial Management Systems (CTMS) for retrieval and transmission of user data related to their participation in clinical research.
Any other repository or database of heath information.
PHMS user data can be stored in the Personal Health Records (PHR) database.

### User Access to Application

Users can gain access to the platform of the invention and its services through any suitable means, for example through a web browser or a mobile device application (also shown in FIG. 1).

With web browser access, the user can navigate to the website and logins of the platform of the invention to begin using the services. For mobile device access to the platform, the user can first download an application to their mobile device. Then the user may access all the platform services through that application on the mobile device.

### Application Features, Modules and Sections

Both the web browser and mobile application accessible services of the platform of the invention can optionally include any or all of the following features:
Health profile
Health insights
Health communities
Secure message center
Activities & Reminders management
Sharing management
Profile management
Upload of medical records via Consolidated Clinical Data Architecture (CCDA) formatted files
Record retrieval using EHR, PPMS, LIMS, and CTMS APIs
Record download into a portable, file format
Automated data mapping using standard clinical codes
Profile management

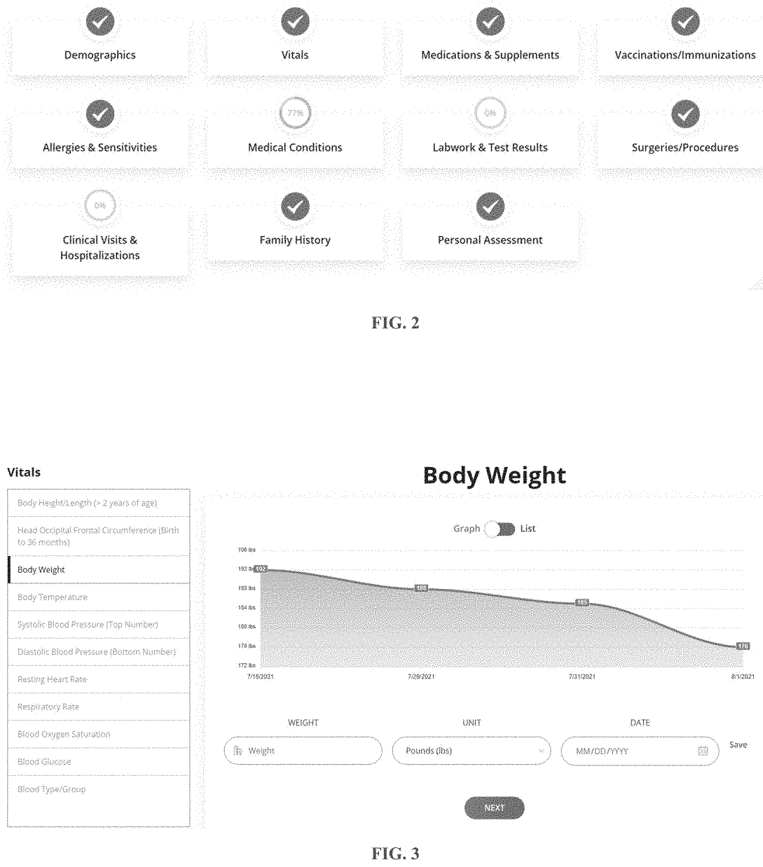

A Health Profile feature (FIG. 2) can be provided and can include any or all of the following modules:
Demographics
Vitals
Medications and Supplements
Vaccinations/Immunizations
Allergies and Sensitivities (Intolerances)
Medical Conditions
Lab Work and Test Results
Surgeries and Procedures
Clinical Visits and Hospitalizations
Family Medical History
Personal Assessment (depression screening tools)

A Health Profile dashboard view can be provided and can show all modules and their respective completion status, for example indicated by a percentage complete or a check-mark symbol once the module has been completed.

A Vitals module can be provided and can include a plurality of health parameters, for example the eleven shown in FIG. 3. Health parameters for inclusion in the Vitals module can be of any suitable type. Suitable health parameters can optionally include parameters that are commonly used indicators of health and which if tracked can provide users insights into their health over time. Additional health parameters can be added to the Vitals module as they become relevant to users. These parameter values may be both manually entered by users, automatically added from uploaded CCDA files or retrieved health records via API or both. The list of vitals can include body height/length, head occipital frontal circumference, body weight, body temperature, systolic and diastolic blood pressure, resting heart rate, respiration rate, blood oxygen saturation, blood glucose, blood type and any other suitable user characteristic or parameter. Vitals can be shown as either a list of entered values over time, as a graph (also shown in FIG. 3) or both.

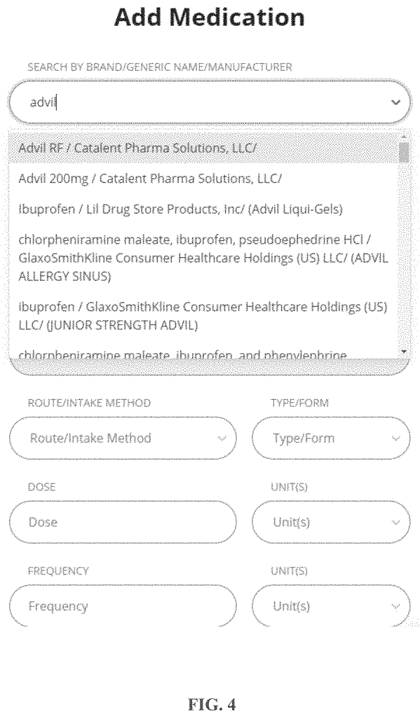

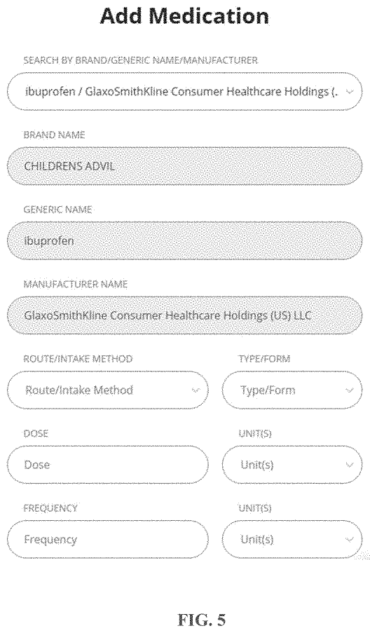

A Medications & Supplements module can be provided and can allow users to add medications to their health profile. Medications may be found in any suitable manner, for example using a search box at the top of the “Add Medications” window (FIG. 4). As the user types either the brand name, generic name, or manufacturer of the medication, the user can be given pull-down menu of options to select. Once selected, the platform of the invention can automatically fill in the BRAND NAME, GENERIC NAME, MANUFACTURER NAME fields and any other desired information (FIG. 5).

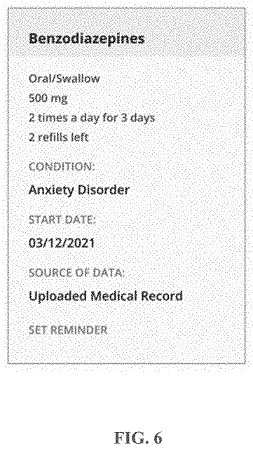

Once a medication is added, it can be displayed as an electronic card in the Medications & Supplements module (FIG. 6). Users may then set a reminder for the medication. The reminder feature can be available for manually entered, uploaded, and retrieved medical record data sources, among possible others.

A Medications & Supplements module can be provided and can have the capability for users to add supplements, for example using a search box function identical to the “Add Medications” window. Setting reminders can also be available for supplements.

A Vaccinations/Immunizations module can be provided and can allow users to add their vaccination history to their health profile. Vaccines may be found for example by using a search box at the top of the “Add Vaccinations/Immunizations” window (FIG. 7). As the user types either the vaccine type or manufacturer, the user can be given pull-down menu of options to select. Once selected, the platform of the invention can automatically fill in the MANUFACTURER NAME field.

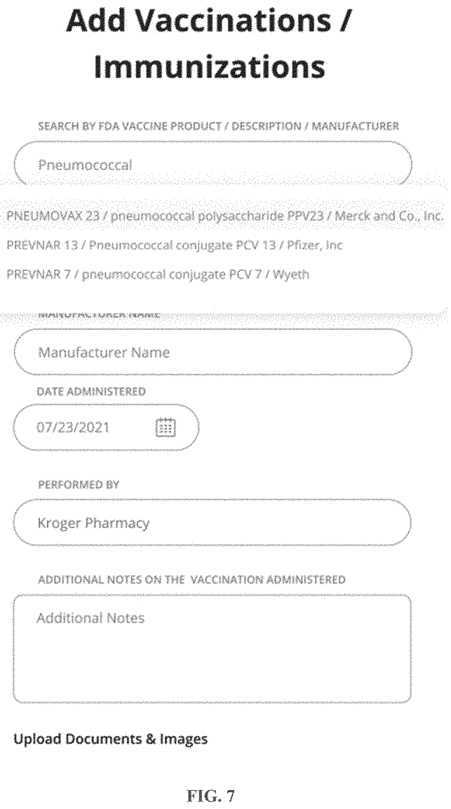

An Allergies and Sensitivities module can be provided and can display both manually entered, uploaded, and retrieved records (FIG. 8), among possible others. Current allergies can be highlighted, for example in green. Former or inactive allergies can be shown in another color, for example grey.

The Medical Conditions module can include the following sections:
Heart, arteries, veins, and circulatory system
Breathing, nose, throat, lungs, and respiratory system
Abnormal growths and cancer
Viruses and infectious diseases
Blood and immune system
Injuries, poison, and physical trauma
Eyes
Ears
Mouth, esophagus, stomach, colon, liver, pancreas, gallbladder, appendix, and digestive system
Thyroid, diabetes, and endocrine system
Skin
Muscles, bones, joints, and ligaments
Nerves and nervous system
Urinary and reproductive systems
Pregnancy, childbirth, and post-childbirth
Newborn injuries relating to the gestation and birth processes
Birth abnormalities and defects
Mental health, neurological health, and developmental challenges
Any other suitable condition

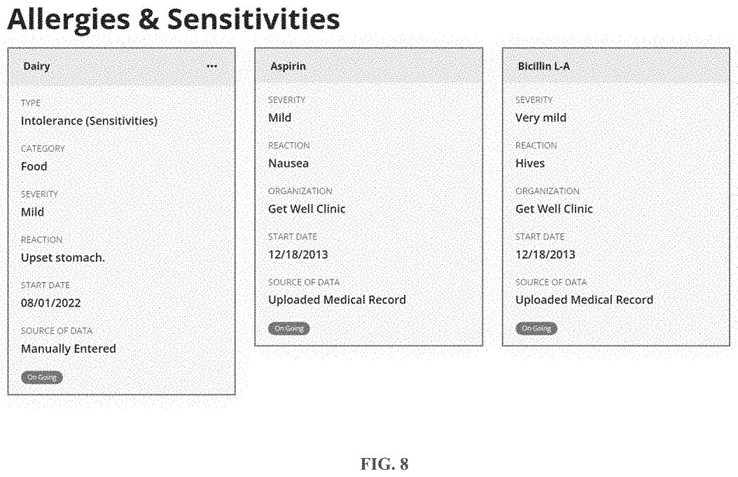

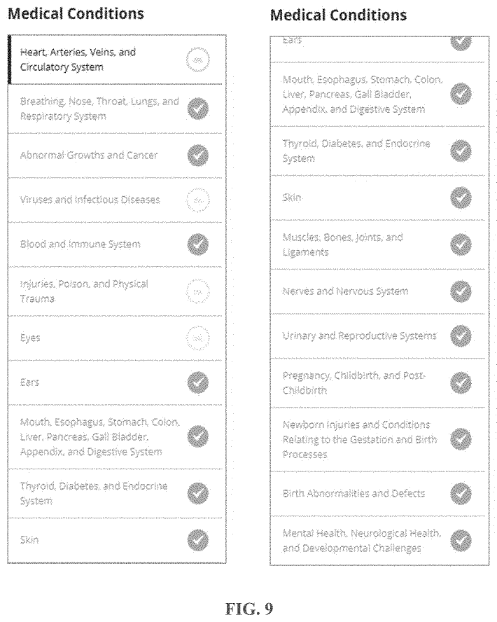

The sections can be selectable features on the Medical Conditions module, for example from a scrollable, sliding list (FIG. 9).

As a section of the module is selected, some potential medical conditions can be displayed to prompt the user to recall their relevant medical history. For each section, the user may select from a list of potential conditions, add a condition that is not listed, or select none. As users review and complete each section with at least one response, the section completion tracking icons can change from a “0%” icon to a check-mark symbol (FIG. 10).

As users select a specific condition, the platform of the invention can prompt the user for the diagnosing healthcare provider, the date of diagnosis, any notes, the option to upload relevant documents and any other suitable option (FIG. 11).

The platform of the invention can display entered, uploaded, and retrieved medical conditions relevant to a section all on one screen (FIG. 12). This can be made possible through the platform's automated data mapping using standard clinical codes.

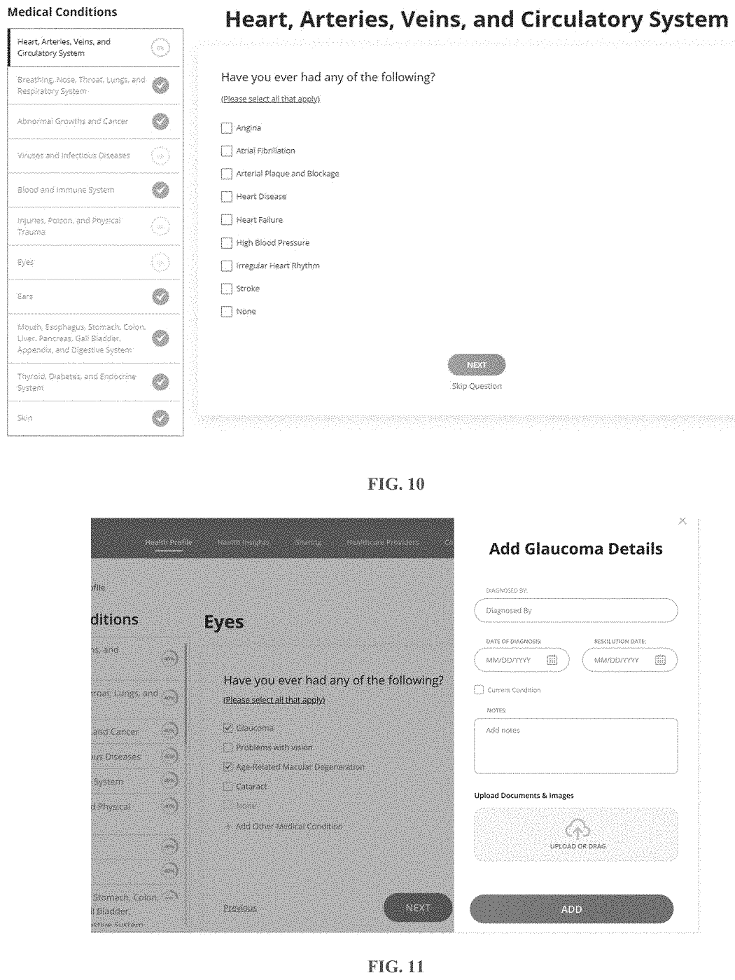

A Lab Work and Test Results module can be provided and can map uploaded and retrieved electronic health records, for example to a running list of lab results (FIG. 13). Electronic lab work and test results that are retrieved for users through file upload or FHIR API can be automatically separated into individual test lines. This can allow users to sort by test type or by date. The normal range for each test can also displayed when available. As needed, users may upload scanned documents of past test results when electronic records are not available.

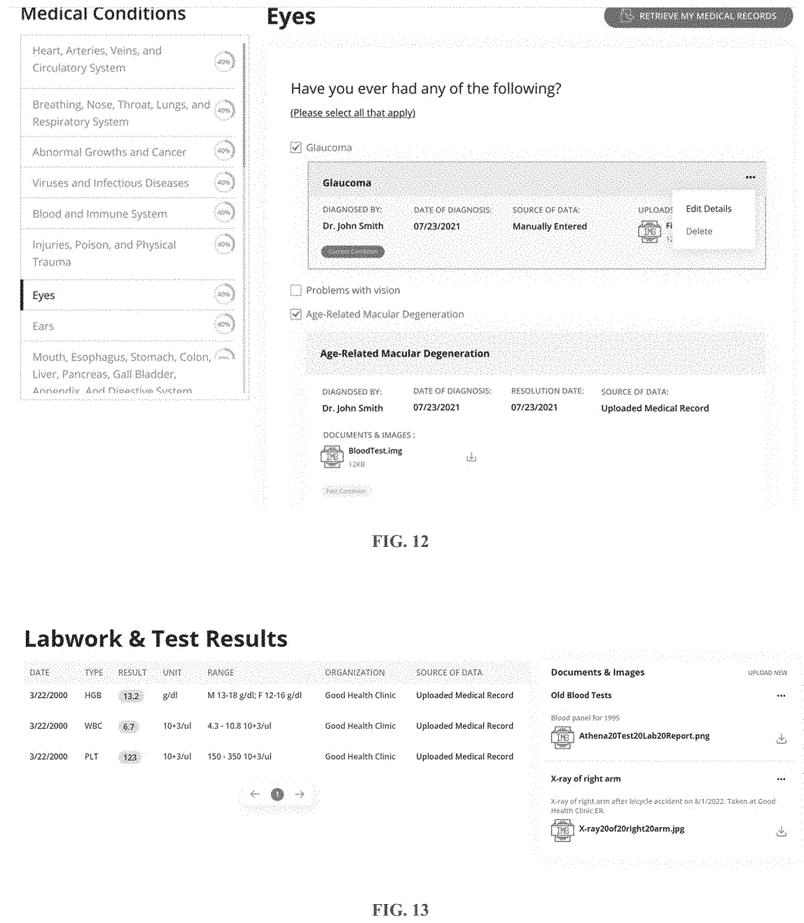

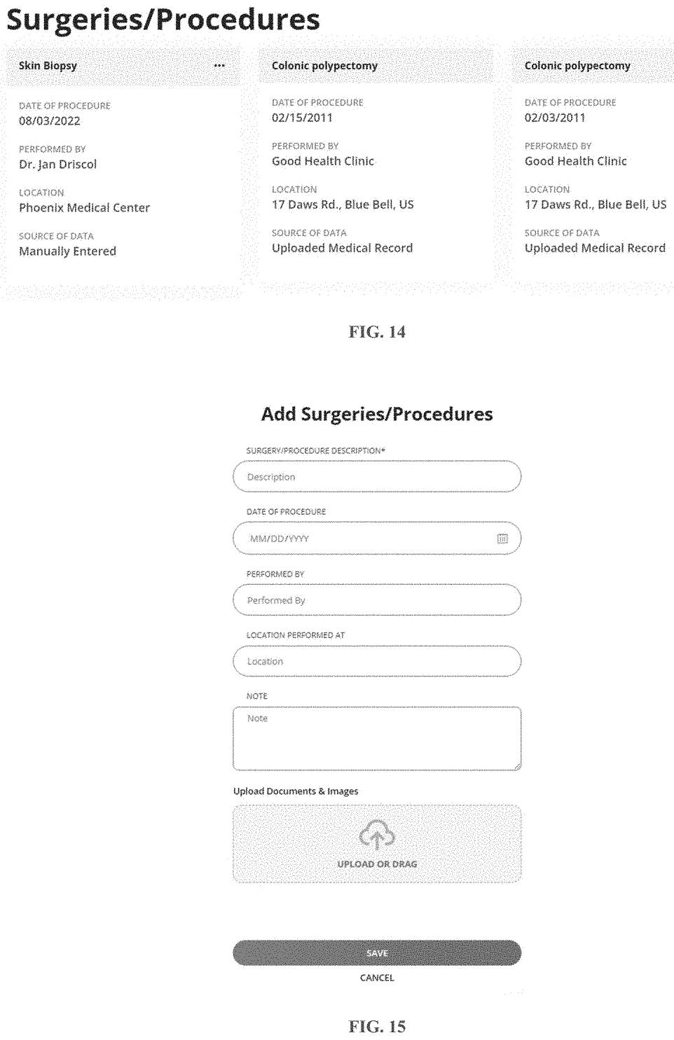

A Surgeries and Procedures module can be provided and can allow users to manually enter past surgeries and items classified as clinical procedures. Electronic Health Records that are uploaded CCDA files or retrieved through FHIR APIs can also be displayed in this module (FIG. 14). Users may also upload scanned documents into the record of a specific surgery and procedure (FIG. 15).

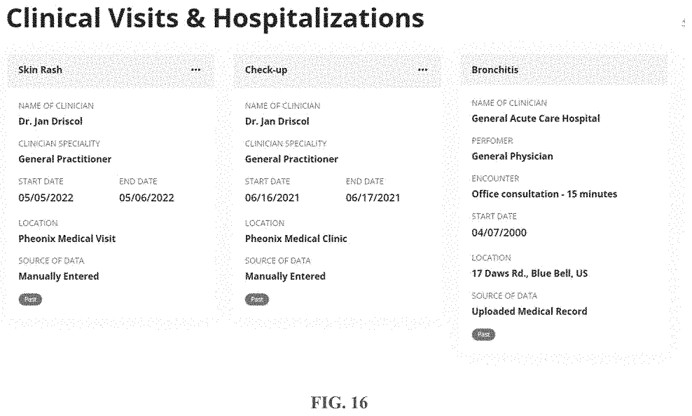

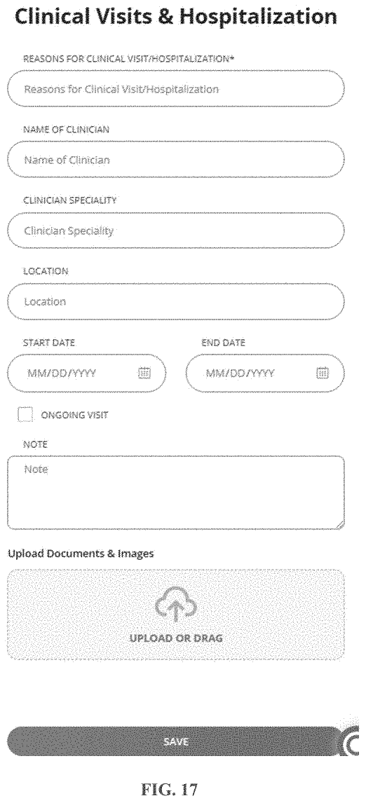

A Clinical Visits and Hospitalizations module can be provided and can allow users to manually enter both past and current clinical visits and hospitalizations. Electronic Health Records that are uploaded CCDA files or retrieved through FHIR APIs can also be displayed in this module (FIG. 16). Users may also upload scanned documents into the record of a clinical visit and hospitalization, for example when entering the details of these clinical encounters visit (FIG. 17).

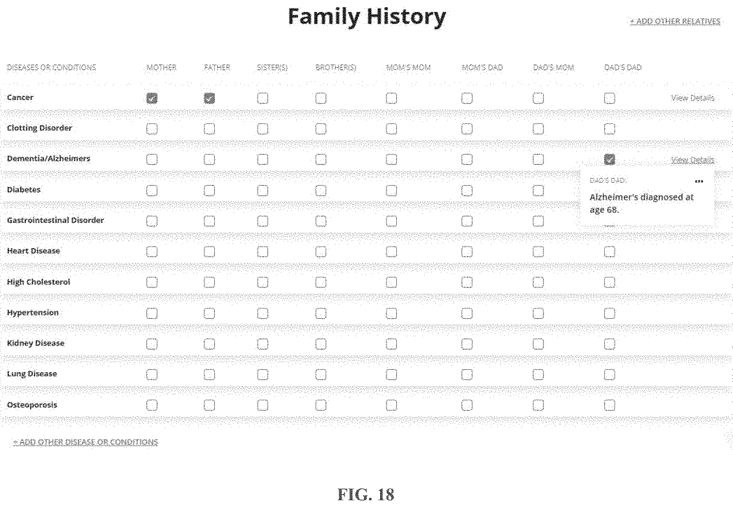

A Family History module can be provided and can be a grid or other suitable graphic or display that allows users to develop and manage their family's entire health history on one screen. It can start with common, for example 11, medical conditions and familial relationships, for example 8 (FIG. 18). Users can click on a box matching a condition to a family member. Then they may add notes related to that family member's specific diagnosis. Additional conditions and family relationships may be added by users.

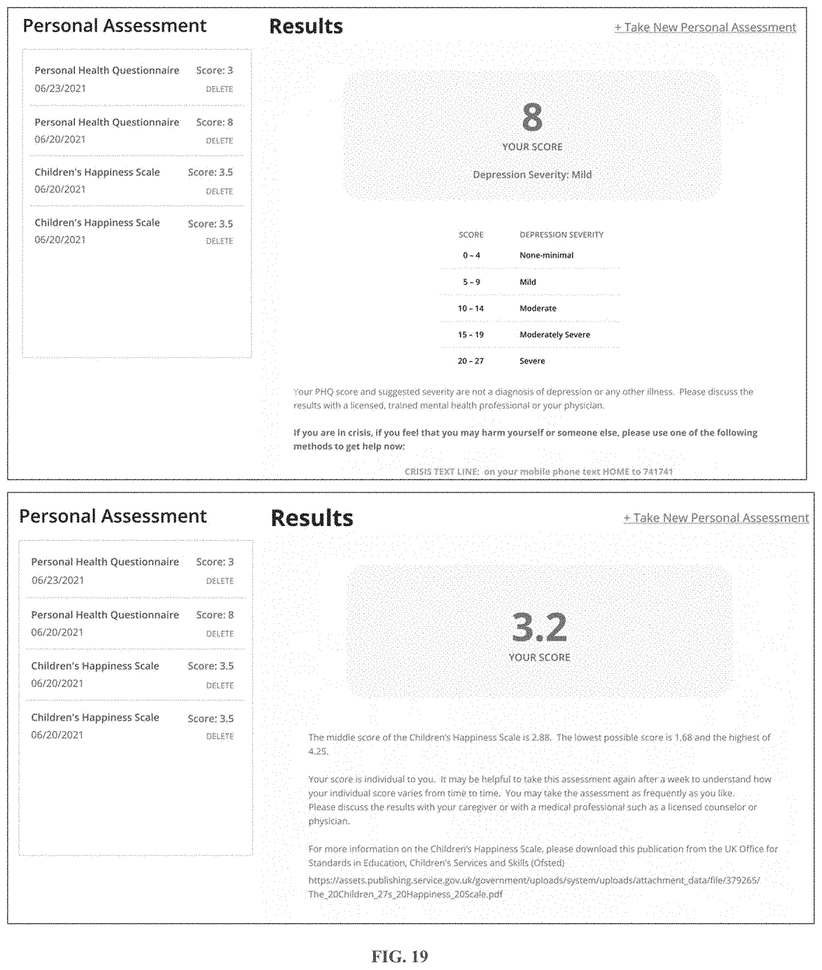

A Personal Assessment module can be provided and can include two depression screening tools with trackable scoring (FIG. 19). One screeding tool can be for adults and another can be for children. The assessments can be made available to the users based on any suitable factor or parameter, for example their age in their profile. The Personal Health Questionnaire can for example be for users over the age of 18 and the Children's Happiness Scale can for example be for users under the age of 18. Other screening tools may be added.

Completion of the Personal Health Questionnaire can generate an instantaneous score and an interpretation with guidance to seek mental health services as advised, for example, by the National Institutes of Health and US Department of Veterans Affairs (VA).

Completion of the Children's Happiness Scale can generate an instantaneous score and provides guidance to seek mental health services as advised, for example, by the National Institutes of Health and the American Academy of Pediatrics.

### Generation of Personalized Health Insights

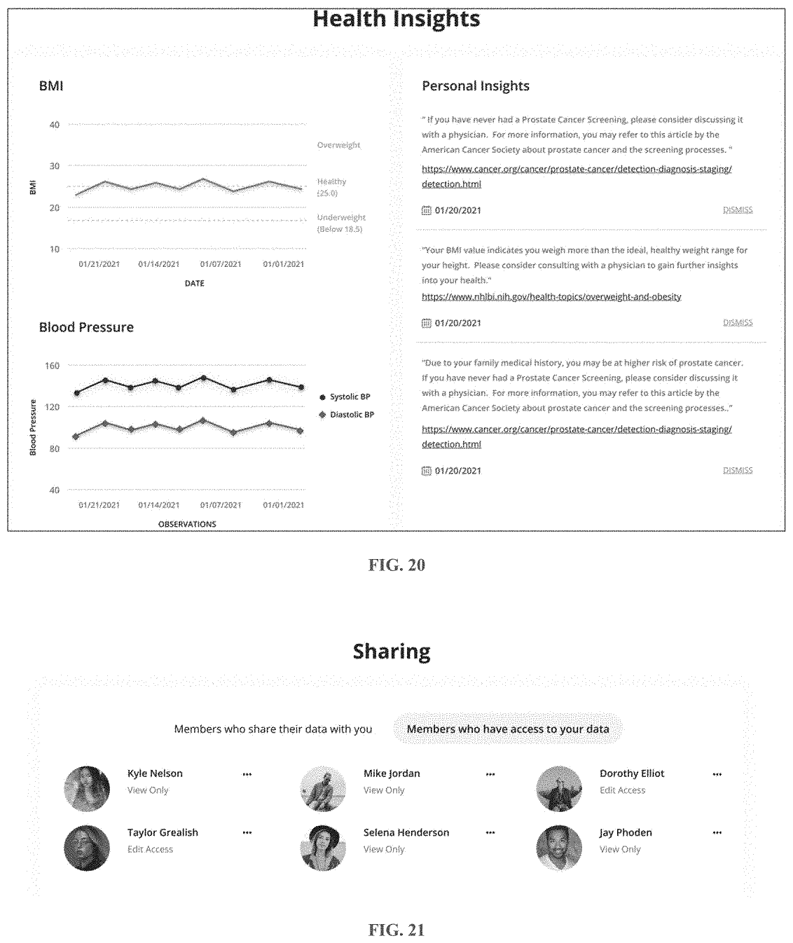

A Health Insights feature can be provided and can deliver personalized insights to the user in any suitable format, for example as either a graph or as personalized messages (FIG. 20).

To generate these health insights, the platform of the invention can use a combination of information from various sources, which can include for example:
Information entered by the user and available in their health profile.
Electronic health records retrieved or uploaded by the user and available in their health profile.
Data collected from the health profiles of all other users of the platform.
Population health statistics and health guidance made available by the Centers for Disease Control and Prevention (CDC), the National Institutes of Health (NIH), the Food and Drug Administration (FDA), the World Health Organization (WHO), UK Office for Standards in Education, Children's Services and Skills (Ofsted), US Department of Veterans Affairs (VA), any other similar agency or any combination of the foregoing.
Diagnostic ranges for medical conditions and diagnostic interpretation guidance made available by the American College of Cardiology, the American Cancer Society, the American Heart Association, the American Academy of Pediatrics, the American Psychological Association, Mental Illness Research Education and Clinical Center (MIRECC), American Diabetes Association, any other similar organization or any combination of the foregoing.
Aggregated patient data made available by hospital systems, insurance companies, pharmacies, pharmaceutical companies, the FDA, clinical research organizations, any other similar organization or any combination of the foregoing.
Historical and real-time environmental data such as air quality, water quality, air temperature, soil studies, and food-borne pathogen alerts, made available through sources such as environmental protection agencies (EPAs), the CDC, the FDA, the National Water Quality Monitoring Council, the National Weather Service, AirNow, any similar public and private organization or any combination of the foregoing.
External factor data collected from users for developing additional health correlations. For example, collecting data on the use of specific products, such as an aerosol air freshener, and a growth in prevalence of a medical condition, such as asthma. This external factor data can include information from the Consumer Product Safety Commission (CPSC) or any similar organizations.

The personal insights can be generated using a combination of statistical and analytical tools including for example the use of artificial intelligence algorithms (AI). For example information or data entered by a user can be analyzed against any, all or some of the foregoing information to generate personal insights for the user.

### Instantaneous Personalized Health Insights

As a user enters information in their health profile, personalized insights can be instantaneously triggered based on data the user entered and an analysis thereof with respect to available medical diagnostic ranges and interpretation guidance.
For example, a personalized health insight can be instantaneously triggered in the following scenarios.
Entry of height and weight can trigger an immediate BMI calculation, including for example with graphs and a Personal Insight message indicating whether that BMI results may be a cause for concern based on CDC guidance.
Entry of a blood pressure reading can trigger a Personal Insight message telling the user whether the reading is within normal range based on guidance from the American College of Cardiology and the American Heart Association.
When graphical charts can be generated based on the data a user enters into their health profile, the chart can optionally show boundary bars to show normal range and abnormal ranges for that particular health parameter.

### Personalized Health Insights Generated Over Time

Personalized insights can be generated when enough information is collected to determine a trend that may be a health concern. The data may be a combination of user data and of some or all available information sources. The platform of the invention can use artificial intelligence algorithms (AI) to periodically scan and analyze all the user data and compare it to all other information sources in order to detect an issue or anomaly in the user's health. The platform can then optionally review available medical guidance to generate recommendations for the user.

For example, a personalized health insight can be generated over time in the following scenarios.
Information from a user's personal health profile, their family medical history, statistical data from the CDC, and diagnostic data from the American Cancer Society can be used by the system, including for example AI of the system, to generate a recommendation for a prostate cancer screening such as a Prostate-Specific Antigen (PSA) Test and Digital Rectal Exam (DRE).
Information about a heart medication a user is currently taking can be combined with data of other users who are also taking the same medication, information from the FDA, information from hospitals, other information or any combination of the foregoing. This combined information can be used by the system, for example AI of the system, to detect a correlation between the heart medication and an increased risk of stroke. The platform or system of the invention can then optionally recommend that the user seek immediate medical advice regarding that heart medication and its risks.
Where users who live near each other are being diagnosed with asthma at a higher rate than is statistically normal as indicated by the CDC and other data sources, the system, for example AI of the system, can detect a correlation between the asthma diagnosis rates and other parameters pertaining to the users, for example user self-reported symptoms or air quality data for those time periods. The system, for example AI of the system, can then review all health profile data for all users within the effected area to determine which users may be susceptible to developing or may have already developed asthma because of the air quality. The platform or system of the invention can then generate recommendations for all affected users providing guidance to seek medical attention and screening for asthma, or some other suitable course of action.

### Physicians Use of Insights

When a user seeks advice from a medical professional, such as a physician, that medical professional may optionally use the platform or system of the invention to assess the user's medical condition and recommend the best treatment for any conditions diagnosed. To generate recommendations to the physician, the platform can optionally use artificial intelligence algorithms (AI) to scan and analyze all the user data and compare it to all other information sources and available medical guidance. Then the system, for example AI of the system, can generate messages for the physician with the suggested diagnoses and recommended treatments, optionally including for example with a statical probability of the accuracy of each diagnosis presented and the potential success of each treatment option.

For example, a user may visit a physician because the user is experiencing a persistent headache. The user can use the platform or system of the invention to grant the physician access to their health profile. Then, when the physician accesses the user's health profile, the platform or system, for example the AI of the platform or system, can automatically generate and highlight relevant medical data to suggest potential diagnoses of the headache, optionally including for example with a percentage probability of accuracy of those diagnoses producing the symptoms the user is experiencing. After the diagnosis is selected by the physician, the platform or system, for example the AI of the platform or system, can recommend treatment options most likely to successfully address the diagnosis, optionally including for example with a statistical success rate presented with each option that is specific to that user's health profile.

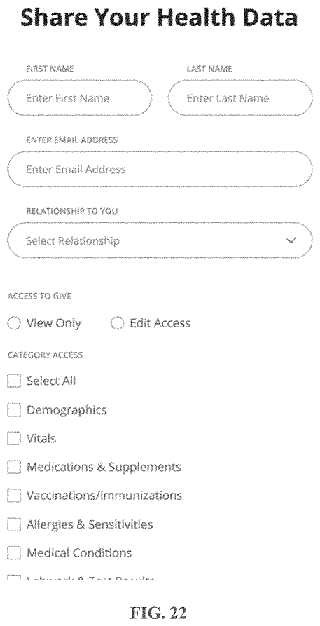

A Sharing feature can be provided and can allow users to pick individual and organizations with whom they may share their data. The status of shared access can be viewable on one screen (FIG. 21). The users may pick which specific features and modules they wish to share (FIG. 22). They may choose to provide edit or view only access.

The Sharing feature can also allow users to share their health data with organizations such as physicians and clinical research organizations.

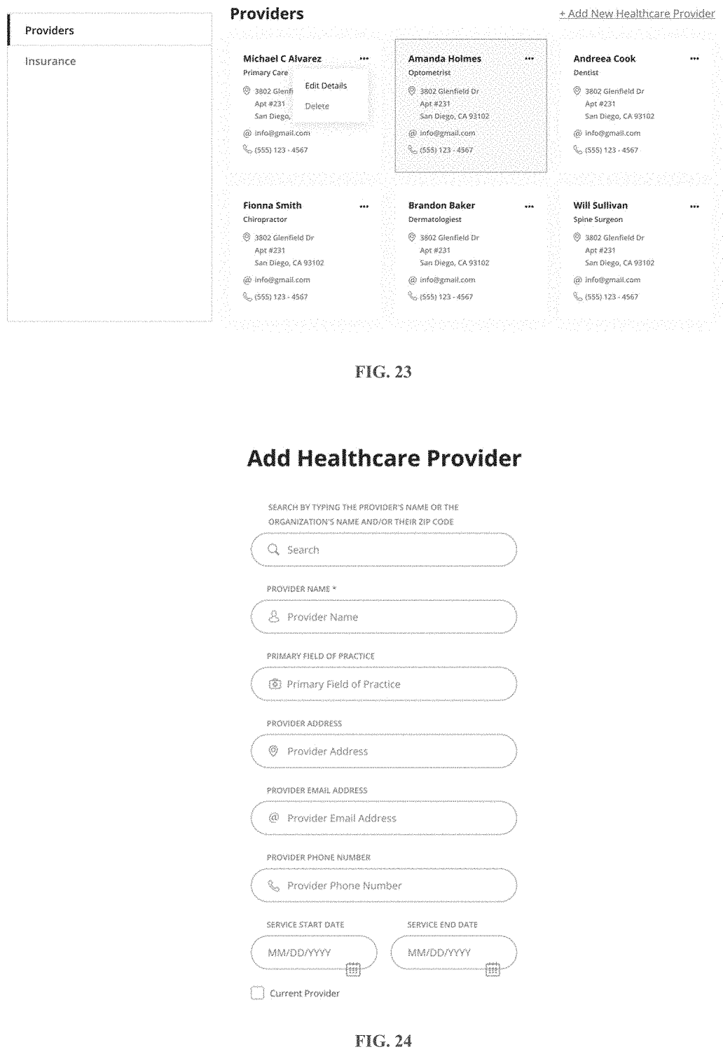

A Provider and Insurance feature can be provided and can allow users to maintain the contact information for their current and past healthcare providers (FIG. 23). It can also allow for the maintenance of health insurance plan information.

When adding healthcare providers, users may search for their Healthcare Providers, for example using the search tool which can validate and retrieve the provider's profile data from the National Plan and Provider Enumeration System (NPPES) registry or any other suitable database (FIG. 24). The NPPES is maintained by the Centers for Medicare & Medicaid Services (CMS) and is access by the platform of the invention through an API. This allows the platform to confirm that the healthcare provider is a valid and registered medical care provider. It also facilitates EHR record retrieval.

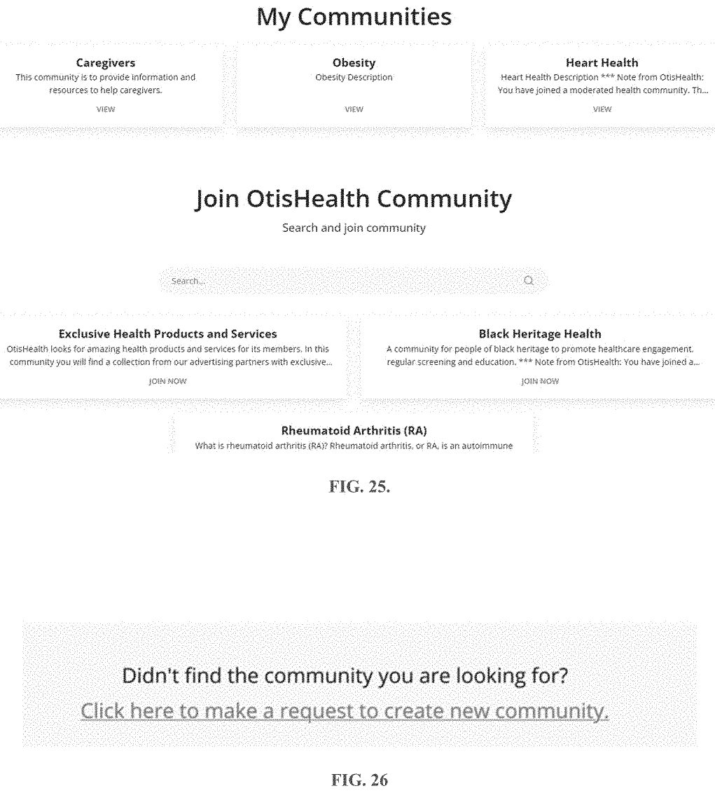

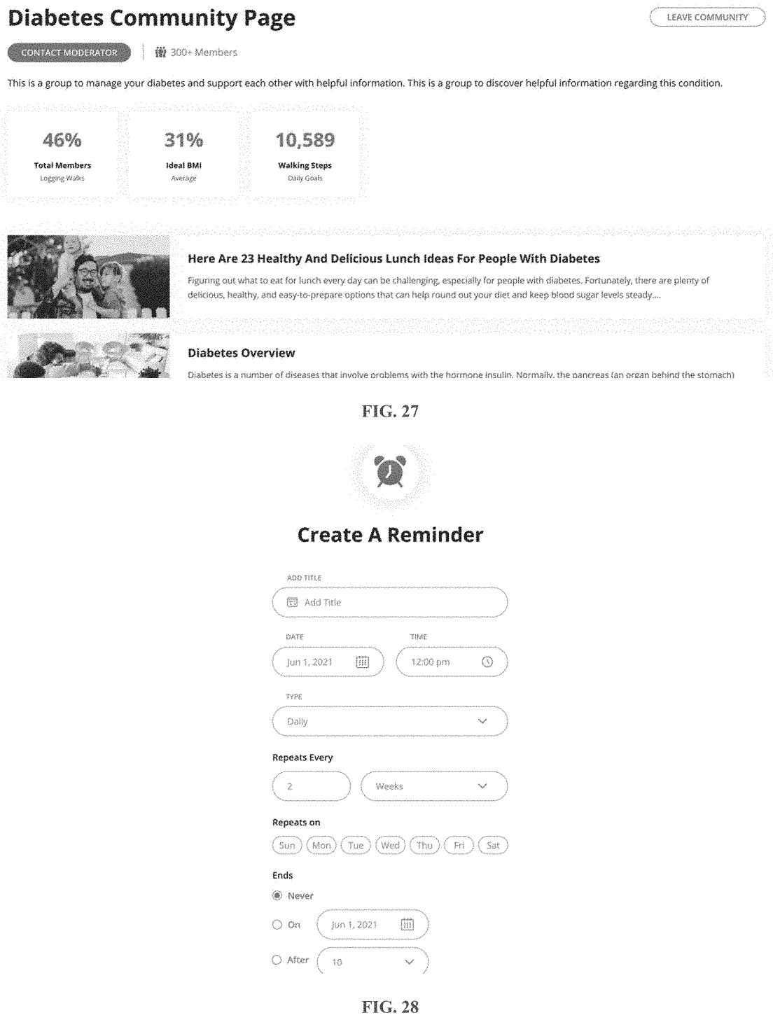

A Communities feature can be provided and can allow users to join existing health communities and view communities which they have already joined (FIG. 25). Users may also request to create a new community (FIG. 26). The community pages can display aggregated community health parameters, metrics, and other content and media relevant to that community (FIG. 27).

An Activity & Reminders feature can be provided and can be used to create any health activity reminder (FIG. 28). Examples of health activities can include exercising, taking medications, completing therapy, taking blood pressure, and scheduling follow-up visits with physicians.

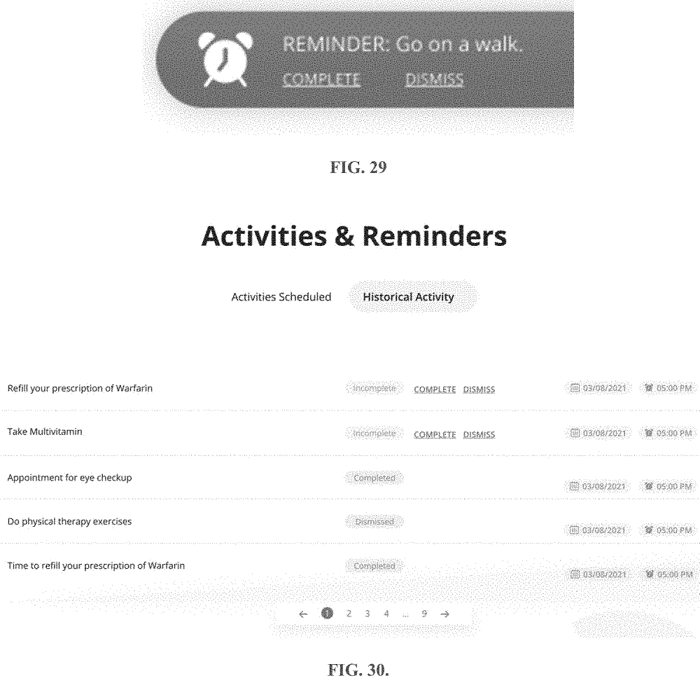

Reminders can appear as an alarm icon on the screen (FIG. 29). When any reminder appears, users can confirm that they completed the activity or dismiss it.

An Activities & Reminders feature can be provided and can allow users to view scheduled activities and track completed and incomplete activities (FIG. 30). All Historical Activity can be timestamped and logged to track user's adherence to their healthcare regiment.

A Health Record Management feature of the platform of the invention can be provided and can allow users to upload Consolidated Clinical Data Architecture (CCDA) formatted files, retrieve healthcare data via APIs, and to download data into a portable file format.

Both when CCDA files are uploaded and when electronic health records are retrieved, the platform of the invention can automatically parse this data within the records and maps the data into the appropriate features, modules, and sections, for example using the platform's automated clinical data mapping capability.

For CCDA upload, the user can select the file location using a file folder browser feature or use a drag-and-drop feature to drag the file into the platform window. If the CCDA file is password-protected, the user may be prompted to enter the password.

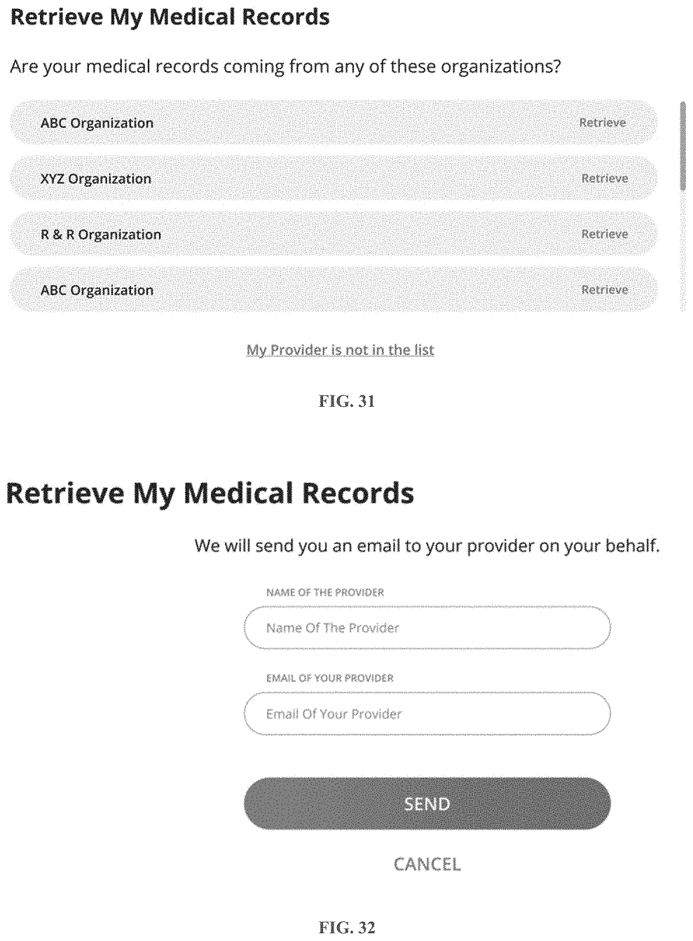

For retrieval of electronic health records using an API, the user can click on the retrieve records button and select the type of record: EHR, PPMS, LIMS, or an insurance record. The user can then be presented a list of organizations, such as hospital systems (FIG. 31).

Once the user selects the organization, the platform of the invention can establish a secure connection to the organization's electronic record system and use the user's data to authenticate the request for the records. All available records for the user can be brought into the platform and loaded into the appropriate features, modules, and sections, for example using the platform's automated clinical data mapping capability.

If the organization is not listed, the user may select an option to manually enter a request to add an additional healthcare provider organization to the list. This request may initiate an automated process to contact the organization and establish a new API connection. The user may also use the platform of the invention to request a CCDA from the organization (FIG. 32). When the organization responds to the email with the properly formatted CCDA file, the platform can read, validate, and load the data into the appropriate features, modules, and sections, for example using the platform's automated clinical data mapping capability.

Users may download portions of the electronic health records from the platform of the invention, for example into a portable CCDA file. The user may select a password or other unique identifier to secure the CCDA file.

### Automated Data Mapping Using Standard Clinical Codes

The Health Profile modules and the sections of the Medical Conditions module can include both manually entered data, user-uploaded electronic health records such as CCDA, electronic health records retrieved using FHIR API (Fast Healthcare Interoperability Resources; Application Programming Interface) connections or any combination of the foregoing.

The platform of the invention can use data mapping or other suitable technology to assign specific standardized clinical diagnostic codes to all medical data. By assigning specific standardized clinical diagnostic codes to all medical data on the platform, all the data can optionally be harmonized, searched and analyzed on a common basis. Specifically, all data entries, whether manually entered, uploaded, or retrieved, can have the same comparative clinical basis for analysis because they will each have a clinical code assigned.

For example, when the platform or system of the invention uses its Artificial Intelligence (AI) capabilities to detect potential health problems based on a user's health information, for example with the AI of the platform or system, the standardized clinical codes that are assigned to all user health data can be used by the AI to more easily classify the data by code category, whether it is a lab result, a medical condition, a medication, or any other classified data. Thus this classification can ensure more efficient and accurate detection of medical concerns.

The standard code set, known as the International Classification of Diseases (ICD), is maintained by the World Health Organization (WHO). As users enter, upload, or retrieve medical records, the platform of the invention can automatically assign and display the health data in a module or section of the platform based on routing shown in Table 1. ICD-9 and ICD-10 are the last two published standards and commonly used for medical coding of clinical activity. The Platform can be able to interpret both ICD-9 and ICD-10 coded records. Records coded as ICD-9 can be automatically converted to the ICD-10 standard for mapping to the proper modules and sections of the platform.

This clinical coding capability can allow for precise placement of user health data within the correct health record modules and sections of the platform of the invention. It can also enable analytical capabilities including the use of artificial intelligence to correlate the users' health data with relevant health concerns such as likelihood of a medical condition, recommendations of clinical diagnostics, and community health alerts.

### Profile Management

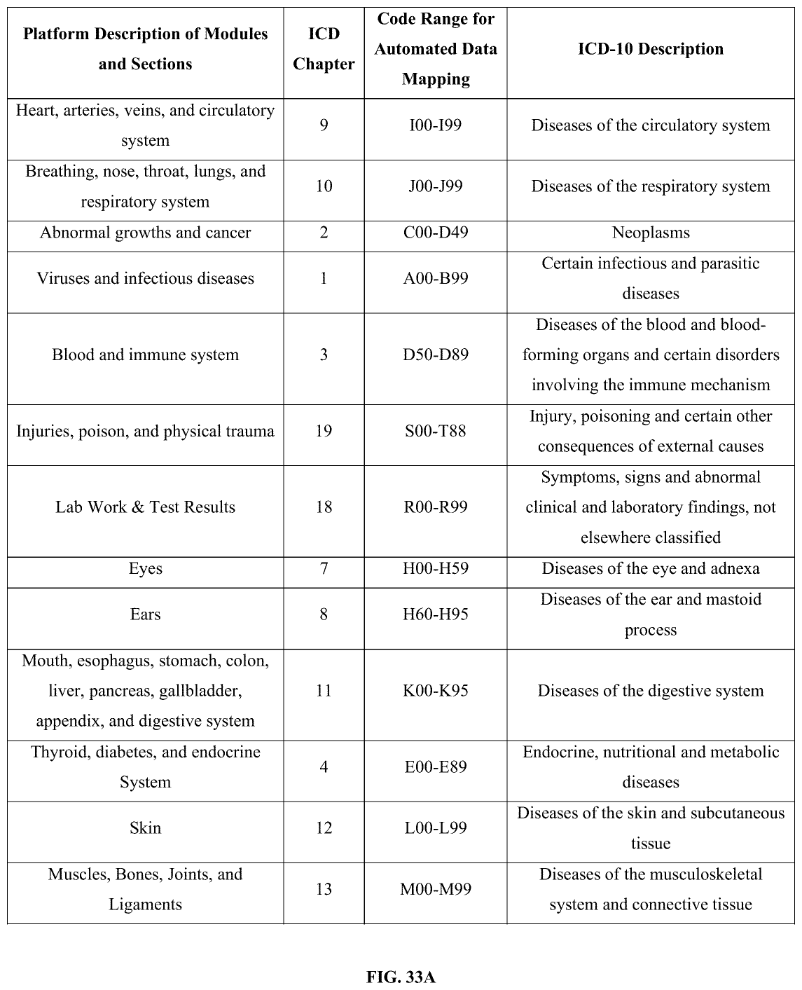

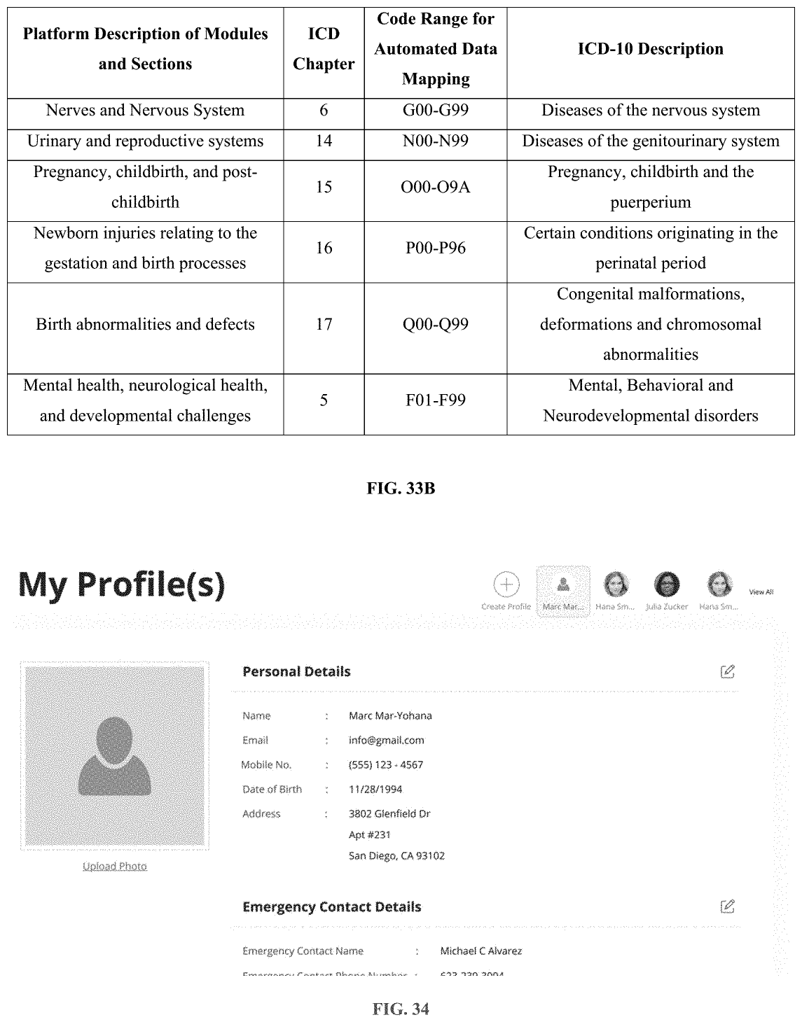

An individual user may establish additional profiles under their profile account (FIG. 33). These additional profiles can be established, for example, for children who are too young to create their own user account or for individuals who have disabilities or limitations that do not allow them the ability to establish and maintain an account.

The additional accounts can be viewable under the primary user's profile management screen

The owner of the primary profile may choose to convert a managed profile into its own, individual account (FIG. 34). This can allow the person within the previously managed profile to create their own login credentials and use all features of the platform of the invention.

### Additional Features for Mobile Device Applications

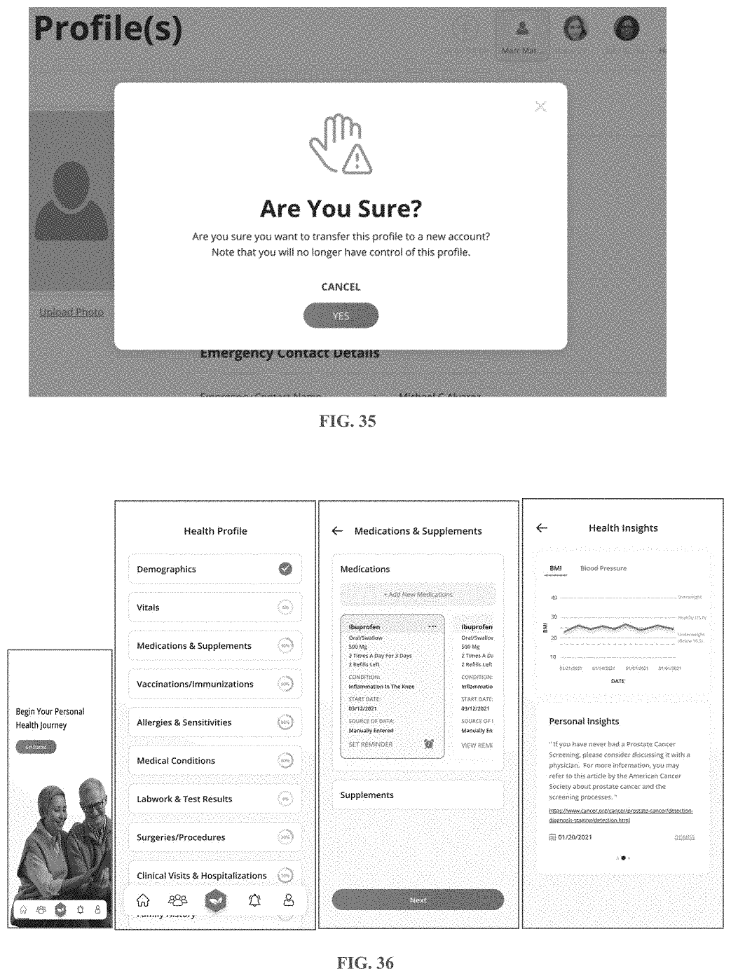

The mobile device application can have all the features of the web-based application (FIG. 35), and optionally added notification and integration capabilities based on the mobile device. Added integration with the mobile device can include:
Visible and audible alerts and reminders, even when the mobile device screen is locked.
Availability of emergency contact information and primary physician contacts.
Availability of critical medical details such as medications, allergies, and blood type.

### Integration with Available Peripheral Devices

The platform of the invention can integrate with third party health, fitness, and wellness applications and any suitable connected peripheral devices, for example such as:
Health activity data captured by the mobile device such as number of steps taken and distance traveled.
Calorie tracking applications.
Activity logging applications.
Heart rate monitoring devices.
Oxygen saturation monitoring devices.
Respiration monitoring devices.
Blood glucose monitoring devices.
Blood pressure monitoring devices.
Thermometers.
Weight scales.
Wearable devices including mobile devices that capture movement, health, and activity data.

This integration can provide automated health data capture that can be recorded to users' personal health record (FIG. 36).

### Common Health Data Representation Through Harmonization, Conditioning and Structure

The platform of the invention creates a common representation of health data collected from all sources that can include data manually entered, data uploaded from electronic health records, data retrieved directly from other systems via API, and data from integrated peripheral devices. This common representation is the result of three system activities.

For the first activity, as data is collected from all sources it is matched to a harmonized numeric unit profile. This ensures that the data and its related unit of measure matches the healthcare data standards used in the platform of the invention for that specific data type. A data type can be blood pressure, blood glucose level, heart rate, and any other standard healthcare data type. Any data that is not matched to a harmonized numeric unit profile is rejected and not added to the user health profile.

For the second activity, the data is converted to the unit of measure used on the platform of the invention for that data type. If the converted and matched number exceeds the variable or numeric range for that data type, the data is rejected and not added to the user health profile. In other words, data that appears to be outside the clinically possible range of values will not be added as it is likely erroneous data.

For the third activity, the data is added in chronological or time-sequenced order with data source tags in one continuous structure per data type. In other words, the data from all sources for an individual data type can be viewed as a continuous list showing each data point, the time the data was registered (measurement was taken), and its corresponding data source. The data source information can include device make, device model, device serial number, software ID, organization name, provider name, location data, and any other information identifying the provenance of the data.

### Community Support Services Coordination Feature

The platform of the invention can be used to coordinate community services and care for users. In certain situations, a person who is seeking healthcare also needs community support services to ensure that their treatment protocol can be completed. Examples of conditions which effect completion of treatment are lack of money, lack of transportation, lack of childcare, lack of safe housing, lack of good nutrition, and other non-medical barriers to treatment. These conditions collectively may also be referred to as Social Determinants of Health (SDoH). SDoH is described by the CDC as conditions in the places where people live, learn, work, and play that affect a wide range of health and quality-of life-risks and outcomes.

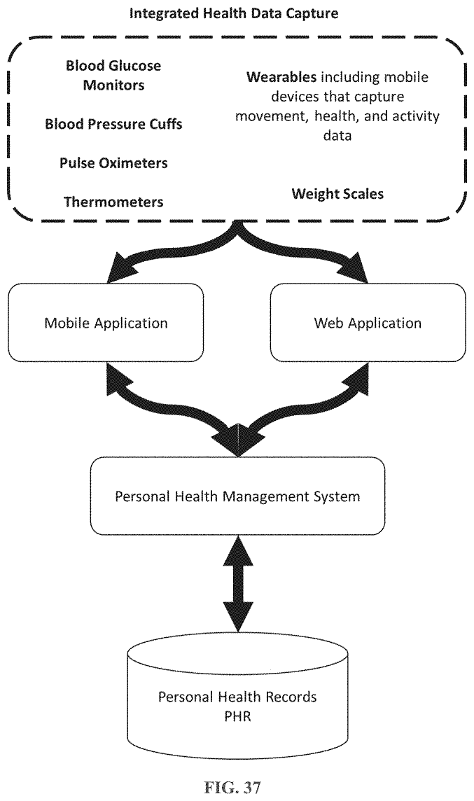

The community coordination feature allows coordination between three groups: healthcare providers, community service organizations, and users who are receiving treatment (FIG. 37).

Users may use the platform of the invention to complete and maintain assessment forms indicating which community services they may require.

Healthcare providers can use the platform of the invention to communicate the care plan to both the patient and the community service organizations. The provider can highlight which community services will help ensure the successful completion of the users treatment protocol.

Community service organizations can use the platform of the invention to review and evaluate the user's needs for services that may facilitate successful completion of the treatment protocol. Community service organizations can use the feature to assign services, track completion, and provide updates to the healthcare providers.

### Special Needs Educational Program Management Feature

The platform of the invention can be used to coordinate the creation and implementation of a child's Individualized Education Plan (IEP). An IEP may consist of at least four components that need to be maintained simultaneously: a physician's diagnosis of a disability, prescribed therapeutic interventions, educational activities, and the directives of the child's parents or legal guardians. An IEP requires the coordination of information between parents/legal guardians, a healthcare provider, an educational institution, and organizations providing therapy or other service support services related to the disability. The platform of the invention can record all elements of an IEP from all contributors: medical diagnosis, documentation of eligibility for services, goals, care plans, therapies, test adaptations, decision logs, calendar of activities, and any other suitable information.

### Caregiver Activity Logs and Schedule

The platform of the invention can be used to log the activities of a caregiver as they provider care for another person. Caregiver Activity Logs can include sections such as Meals, Hydration, Toilet Observations, Movement and Physical Activity, Personal Hygiene, General Notes, and any other suitable information. The platform of the invention can also provide a scheduling tool such as a calendar that caregivers may use to schedule their arrival, departure, care activities, and any other suitable event.

### User Consent to Send Records to a Healthcare Provider

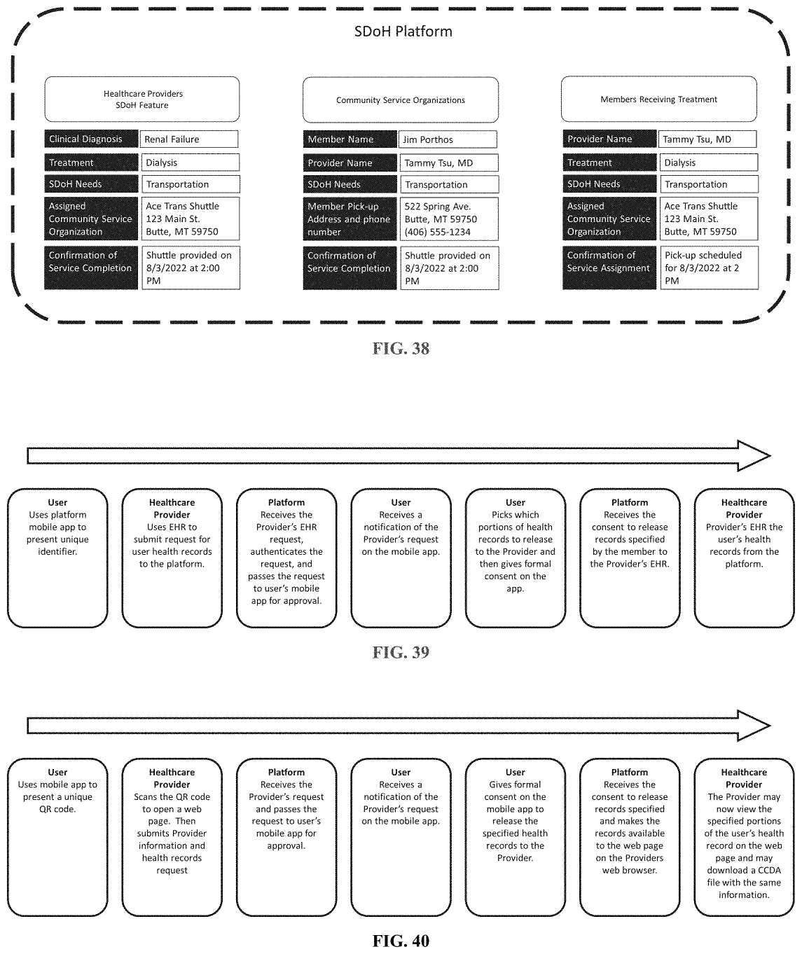

During the admission or health evaluation process (FIG. 38) at a healthcare provider users can use the platform of the invention to consent to have the platform to transfer all or a portion of the users' electronic health records directly to the provider's EHR.

When the user presents themselves at a provider, they will inform the provider staff that they are users of platform of the invention and provide their platform unique identifier. The provider staff will then use their EHR to initiate an API call to the platform FHIR endpoint with a request to retrieve the user's health records using the platform unique identifier.

The platform of the invention will alert the user on their platform application that a request has been made to access their electronic health records. The user can then use the platform application to approve the portions of their medical records they wish to have released to the provider. Once the user approves the portions to release, the platform will send the electronic records to the provider's EHR.

A record of the request, consent, and transfer of records will be maintained on the platform of the invention.

### Alternative User Consent to Make Electronic Health Records Available to a Healthcare Provider

During the admission or health evaluation process (FIG. 40) at a healthcare provider, Users can use the mobile app to consent to have the platform of the invention to transfer all or a portion of the User's electronic health records directly to the provider via a CCDA file or other file transfer.

When the User presents themselves at a provider, they will inform the provider staff that they are users of the platform or system of the invention and use their Mobile application to display their health record consent QR code. The QR code includes a time-limited Uniform Resource Locator (URL) or web address that includes a string of data unique to that user and an expiring string of access numbers (Access Token). The Provider staff will scan the QR code using a camera or other scanning means. The QR code will open a web page using a web browser on a computer or other device capable of supporting a web browser. The Provider staff will have the option to enter their name, the name of the organization, their contact information, any other relevant provider details, and then select the portions of the user's health record they wish to receive.

Once the provider staff have submitted the information on the web page, the platform will alert the User on their Mobile application that a request has been made to access their electronic health records, who made the request, and what was requested. The User can then use the Mobile application to approve the portions of their medical records they wish to have released to the provider. Once the User approves the portions to release, the platform will make the information available to the provider on the webpage as a page-viewable record and as a downloadable CCDA file.

A record of the request, consent, and transfer of records will be maintained on the platform or system of the invention.

### Sample Applications

In some aspects of the invention, a computerized method for analyzing healthcare data of a user can be provided that includes uploading electronic healthcare data of the user into a system, the electronic healthcare data including a first plurality of healthcare events of the user, manually entering a second plurality of healthcare events of the user into the system, each event in the first and second plurality of healthcare events including at least a date, medical data pertaining to the user and a source of the medical data, assigning each event in the first and second plurality of healthcare events to at least one medical category and ordering the events in each medical category by date whereby the aggregation of electronic and manual healthcare events and chronological ordering of the events facilitates diagnosis and treatment of the user.

The method can optionally further include searching the first and second plurality of healthcare events with respect to a specified health issue and causing the system to display first occurrence information with respect to the specified health issue. The electronic healthcare data can optionally include electronic data selected from the group consisting of data from a wearable device of the user, data from a home health monitoring device of the user, electronic health records data, electronic health records data retrieved via FHIR API and any combination of the foregoing. The at least one medical category can optionally be selected from the group consisting of demographics, vitals, medications, supplements, vaccinations, immunizations, allergies, sensitivities, medical conditions, labwork, test results, surgeries, procedures, clinical visits, hospitalizations, family history, personal assessment and any combination of the foregoing.

In some aspects of the invention, a computerized method for a user to consent to release of electronic health records of the user can be provided that includes causing a computerized system to generate a unique system identifier of the user upon a request of the user by means of a computing device of the user, electronically transmitting the identifier to a computer network of a healthcare provider requesting the electronic health records, upon request of the healthcare provider transmitting the identifier from the computer network of the healthcare provider to a computer network that is storing the electronic health records, causing the computer network that is storing the electronic health records to send an electronic request to the computing device of the user to consent to the release of a user determined portion of the electronic health records to the healthcare provider.

The computing device of the user can optionally be a smartphone. The transmitting of the identifier from the computer network of the healthcare provider to the computer network that is storing the electronic health records of the user can optionally include transmitting the identifier via FHIR API. The causing the computer network that is storing the electronic health records of the user to send an electronic request to the computing device of the user to consent to the release of a user determined portion of the electronic health records of the user to the healthcare provider can optionally include sending the electronic request via FHIR API. The identifier can optionally be a time-limited identifier.

In some aspects of the invention, a computerized method for a user to consent to release of electronic health records of the user can be provided that includes causing a first screen to open on a computing network of a healthcare provider upon the healthcare provider scanning a unique QR code provided by the user, the screen being configured to permit the healthcare provider to select portions of the electronic health records for release to the healthcare provider and submit a release request for selected portions of the electronic health records, transmitting the release request from the computer network of the healthcare provider to a computer network that is storing the electronic health records, causing the computer network that is storing the electronic health records to send the release request to a computing device of the user to consent to the release of a user determined portion of the selected portions of the electronic health records to the healthcare provider, upon receiving a consent from the user causing a second screen to open on a computing device of the healthcare provider for permitting the healthcare provider to obtain access to the selected portions of the electronic health records.

The web browser can optionally be prepopulated with user details. The web browser can optionally be configured to require the name of the person requesting the electronic health record on behalf of the healthcare provider. Each of the first screen and the second screen can optionally be a web browser. The permitting the healthcare provider to obtain access to the selected portions of the electronic health records can optionally include permitting the healthcare provider to take an action selected from the group consisting of viewing the selected portions of the electronic health records and downloading the selected portions of the electronic health records.

In some aspects of the invention, a computerized method for a user to consent to release of electronic health records of the user can be provided that includes computing device of a user to generate a unique QR code that contains details of a unique computing session for accessing the electronic health records and causing the unique computing session to open on a computer network of a healthcare provider upon the healthcare provider scanning the unique QR code, the unique computing session including a temporary connection to a computer network that is storing the electronic health records.

The unique computing session can optionally be a unique web browser session. The unique computing session can optionally be time limited. The unique QR code can optionally contain details of a unique, single computing session for accessing the electronic health records.

We claim:

1. A computerized method for analyzing healthcare data of a user, comprising uploading electronic healthcare data of the user into a system, the electronic healthcare data including a first plurality of healthcare events of the user, manually entering a second plurality of healthcare events of the user into the system, each event in the first and second plurality of healthcare events including at least a date, medical data pertaining to the user and a source of the medical data, assigning each event in the first and second plurality of healthcare events to at least one medical category and ordering the events in each medical category by date whereby the aggregation of electronic and manual healthcare events and chronological ordering of the events facilitates diagnosis and treatment of the user.

2. The method of claim 1, further comprising searching the first and second plurality of healthcare events with respect to a specified health issue and causing the system to display first occurrence information with respect to the specified health issue.

3. The method of claim 1, wherein the electronic healthcare data includes electronic data selected from the group consisting of data from a wearable device of the user, data from a home health monitoring device of the user, electronic health records data, electronic health records data retrieved via FHIR API and any combination of the foregoing.

4. The method of claim 1, wherein the at least one medical category is selected from the group consisting of demographics, vitals, medications, supplements, vaccinations, immunizations, allergies, sensitivities, medical conditions, labwork, test results, surgeries, procedures, clinical visits, hospitalizations, family history, personal assessment and any combination of the foregoing.

5. A computerized method for a user to consent to release of electronic health records of the user, comprising causing a computerized system to generate a unique system identifier of the user upon a request of the user by means of a computing device of the user, electronically transmitting the identifier to a computer network of a healthcare provider requesting the electronic health records, upon request of the healthcare provider transmitting the identifier from the computer network of the healthcare provider to a computer network that is storing the electronic health records, causing the computer network that is storing the electronic health records to send an electronic request to the computing device of the user to consent to the release of a user determined portion of the electronic health records to the healthcare provider.

6. The method of claim 5, wherein the computing device of the user is a smartphone.

7. The method of claim 5, wherein the transmitting of the identifier from the computer network of the healthcare provider to the computer network that is storing the electronic health records of the user includes transmitting the identifier via FHIR API.

8. The method of claim 5, wherein the causing the computer network that is storing the electronic health records of the user to send an electronic request to the computing device of the user to consent to the release of a user determined portion of the electronic health records of the user to the healthcare provider includes sending the electronic request via FHIR API.

9. The method of claim 5, wherein the identifier is a time-limited identifier.

10. A computerized method for a user to consent to release of electronic health records of the user, comprising causing a first screen to open on a computing network of a healthcare provider upon the healthcare provider scanning a unique QR code provided by the user, the screen being configured to permit the healthcare provider to select portions of the electronic health records for release to the healthcare provider and submit a release request for selected portions of the electronic health records, transmitting the release request from the computer network of the healthcare provider to a computer network that is storing the electronic health records, causing the computer network that is storing the electronic health records to send the release request to a computing device of the user to consent to the release of a user determined portion of the selected portions of the electronic health records to the healthcare provider, upon receiving a consent from the user causing a second screen to open on a computing device of the healthcare provider for permitting the healthcare provider to obtain access to the selected portions of the electronic health records.

11. The method of claim 10, wherein the web browser is prepopulated with user details.

12. The method of claim 10, wherein the web browser is configured to require the name of the person requesting the electronic health record on behalf of the healthcare provider.

13. The method of claim 10, wherein each of the first screen and the second screen is a web browser.

14. The method of claim 10, wherein the permitting the healthcare provider to obtain access to the selected portions of the electronic health records includes permitting the healthcare provider to take an action selected from the group consisting of viewing the selected portions of the electronic health records and downloading the selected portions of the electronic health records.

15. A computerized method for a user to consent to release of electronic health records of the user, comprising a computing device of a user to generate a unique QR code that contains details of a unique computing session for accessing the electronic health records and causing the unique computing session to open on a computer network of a healthcare provider upon the healthcare provider scanning the unique QR code, the unique computing session including a temporary connection to a computer network that is storing the electronic health records.

16. The method of claim 15, wherein the unique computing session is a unique web browser session.

17. The method of claim 15, wherein the unique computing session is time limited.

18. The method of claim 15, where in the unique QR code contains details of a unique, single computing session for accessing the electronic health records.
